(19) United States
(12) Patent Application Publication
(10) Pub. No.: US 2024/0210385 A1
(43) Pub. Date: Jun. 27, 2024

(54) METHODS FOR CHARACTERIZING LENTIVIRUSES

(71) Applicant: LONZA HOUSTON, INC., Pearland, TX (US)

(72) Inventors: Vijetha BHAT, Pearland, TX (US); Peng WANG, Pearland, TX (US); Chien-Ting LI, Pearland, TX (US); Bingnan GU, Pearland, TX (US)

(21) Appl. No.: 18/480,841

(22) Filed: Oct. 4, 2023

Related U.S. Application Data
(60) Provisional application No. 63/476,711, filed on Dec. 22, 2022.

(51) Int. Cl. G01N 33/543; C07K 16/10; C07K 16/40; G01N 21/64

(52) U.S. Cl. CPC G01N 33/543; C07K 16/1054; C07K 16/40; G01N 21/64; G01N 2021/6439; G01N 2333/155

(57) ABSTRACT

A method of characterizing a lentivirus, comprising: providing a sample comprising a lentivirus population comprising a fully loaded lentivirus, a partially loaded lentivirus, and/or an empty lentivirus; contacting a substrate with the sample to capture the lentivirus population; contacting the captured lentivirus population with a first fluorescent agent comprising a first fluorescent label and a first binding molecule that binds an envelope protein of the lentivirus, a second fluorescent agent comprising a second fluorescent label and a second binding molecule that binds a capsid protein of the lentivirus, and a third fluorescent agent comprising a third fluorescent label that binds a payload; illuminating the captured lentivirus population with light to excite the fluorescent agents; detecting fluorescent lights emitted from the fluorescent agents at different wavelengths; and characterizing the lentivirus according to the detected fluorescent lights.

### FIELD OF THE INVENTION

The present disclosure provides methods for characterizing a lentiviral vector using multiple fluorescent agents that respectively bind to an envelope protein, a capsid protein, and/or a payload of the lentiviral vector. By detecting the fluorescence at different wavelengths, fully loaded lentiviruses, partially loaded lentiviruses, and empty lentiviruses can be identified and quantified. The methods provide rapid, simple, and simultaneous characterization of lentiviral vectors, which aids optimization of lentiviral vector yield.

### BACKGROUND OF THE INVENTION

Lentiviral vectors are biological vehicles that can be used to deliver therapeutic genes (“payload”) into cells. Current lentiviral manufacturing processes typically generat only ˜20% fully loaded lentiviral vectors containing the three viral components, and the payload. The high amount of empty or partially loaded lentiviral vectors reduces the efficacy of lentivirus-mediated gene delivery while increasing the immunogenic burden in the transduced cells ex vivo or in vivo, which can trigger serious adverse events in patients. What is needed is a method of identifying lentiviral vectors having complete loading, as well as partially loaded vectors. The present invention fulfills this need.

### SUMMARY OF THE INVENTION

In some embodiments, provided herein is a method of characterizing a lentiviral vector, comprising: providing a sample comprising a lentiviral vector population comprising a fully loaded lentiviral vector, a partially loaded lentiviral vector, and/or an empty lentiviral vector; contacting a substrate with the sample, thereby capturing the lentiviral vector population on the substrate, wherein the substrate comprises anti-envelope protein antibodies; contacting the captured lentiviral vector population with a first fluorescent agent comprising a first fluorescent label and a first binding molecule that binds an envelope protein of the lentiviral vector, a second fluorescent agent comprising a second fluorescent label and a second binding molecule that binds a capsid protein of the lentiviral vector, and a third fluorescent agent comprising a third fluorescent label that binds a payload of the lentiviral vector; illuminating the captured lentiviral vector population with light from an illumination source, thereby exciting the first fluorescent agent, the second fluorescent agent, and the third fluorescent agent; detecting a first fluorescent light at a first fluorescent wavelength emitted from the first fluorescent agent, a second fluorescent light at a second fluorescent wavelength emitted from the second fluorescent agent, and a third fluorescent light at a third fluorescent wavelength emitted from the third fluorescent agent; and characterizing the lentiviral vector according to the detected first, second, and third fluorescent lights, wherein the first, second, and third fluorescent wavelengths are different.

### BRIEF DESCRIPTION OF THE DRAWINGS

FIG. 1 shows the structure of a lentiviral vector having a payload.

FIG. 2 shows a method of three-layer visualization of lentiviral vectors according to some embodiments of the present disclosure.

FIG. 3 shows packaging plasmids, envelope plasmid and transfer plasmid for producing lentiviral vectors according to some embodiments of the present disclosure.

FIG. 4 shows different types of capsid loading of recombinant adeno-associated virus (rAAV) in rAAV production.

FIG. 5 shows lentiviral vector profiles produced using three different ratios of plasmids according to some embodiments of the present disclosure.

FIG. 6 shows lentiviral vector profiles produced using five different ratios of plasmids according to some embodiments of the present disclosure, which expresses green fluorescent protein (GFP).

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps.

As used herein, “nucleic acid,” “nucleic acid molecule,” or “oligonucleotide” means a polymeric compound comprising covalently linked nucleotides. The term “nucleic acid” includes polyribonucleic acid (RNA) and polydeoxyribonucleic acid (DNA), both of which may be single- or double-stranded. DNA includes, but is not limited to, complimentary DNA (cDNA), genomic DNA, plasmid or vector DNA, and synthetic DNA. RNA includes, but is not limited to, mRNA, tRNA, rRNA, snRNA, microRNA, miRNA, or MIRNA.

A “gene” as used herein refers to an assembly of nucleotides that encode a polypeptide, and includes cDNA and genomic DNA nucleic acid molecules. “Gene” also refers to a nucleic acid fragment that can act as a regulatory sequence preceding (5′ non-coding sequences) and following (3′ non-coding sequences) the coding sequence. In some embodiments, genes are integrated with multiple copies. In some embodiments, genes are integrated at predefined copy numbers.

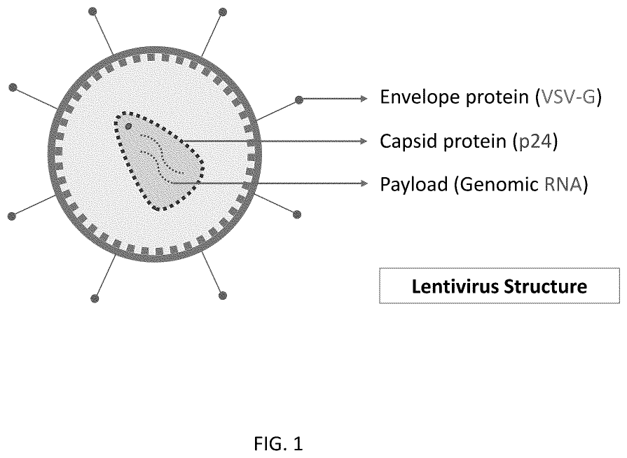

As shown in FIG. 1, a functional lentiviral vector (also called functional lentivirus herein) has three critical components—an outer envelope comprised of, for example, vesicular somatitis virus glycoprotein (VSV-G) protein; a capsid housing the genetic material comprised of, for example, p24 protein; and a payload and/or genomic RNA, oftena therapeutic nucleic acid. Currently there is no single assay that gives a thorough picture of the lentiviral vector following production. Current assays are time-consuming, low-throughput and often, labor-intensive. In addition, most assays only measure a single attribute of the lentiviral vector, and not the complete vector structure.

In certain aspects, the present disclosure provides a method of characterizing lentiviral vectors using three-layer visualization. In some embodiments, the method does not require purification of the lentiviral vector from the culture medium, and can be performed at high-throughput to analyze multiple samples simultaneously.

In some embodiments, the method of characterizing the lentiviral vector includes: providing a sample comprising a lentiviral vector population comprising a fully loaded lentiviral vector, a partially loaded lentiviral vector, and/or an empty lentiviral vector; contacting a substrate with the sample, thereby capturing the lentiviral vector population on the substrate, wherein the substrate comprises anti-envelope protein antibodies; contacting the captured lentiviral vector population with a first fluorescent agent comprising a first fluorescent label and a first binding molecule that binds an envelope protein of the lentiviral vector, a second fluorescent agent comprising a second fluorescent label and a second binding molecule that binds a capsid protein of the lentiviral vector, and a third fluorescent agent comprising a third fluorescent label that binds a payload. The method further comprises illuminating the captured lentivirus population with light from an illumination source, thereby exciting the first fluorescent agent, the second fluorescent agent, and the third fluorescent agent. The method further includes detecting a first fluorescent light at a first wavelength emitted from the first fluorescent agent, a second fluorescent light at a second wavelength emitted from the second fluorescent agent, and a third fluorescent light at a third fluorescent wavelength emitted from the third fluorescent agent; and then characterizing the lentivirus according to the detected first, second, and third fluorescent lights. In some embodiments, the first, second, and third fluorescent wavelengths are different.

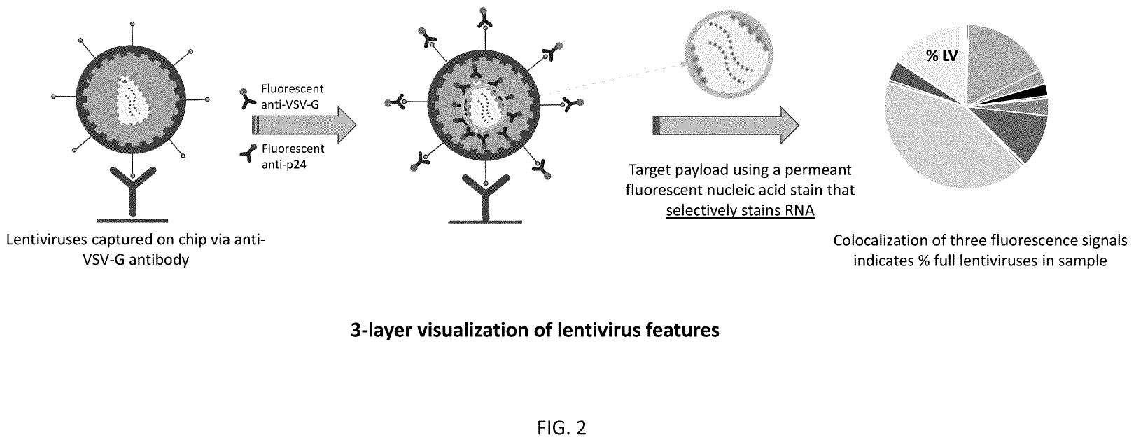

FIG. 2 shows a method of three-layer visualization of lentiviral vectors according to some embodiments of the present disclosure, where fluorescent anti-VSV-G antibodies, fluorescent anti-p24 antibodies, and a fluorescent nucleic acid stain are used to provide the three-layer visualization. In some embodiments, the present disclosure targets the payload by using a permeant fluorescent nucleic acid stain that selectively stains the RNA payload.

Suitably lentivirus samples are contacted with a substrate, for example a microarray chip that includes envelope-capture antibodies such as anti VSV-G capture antibodies. As shown in FIG. 2, the capture antibodies capture lentiviral vectors via the interaction between surface-bound antibodies and antibodies on the outside of the lentiviral vectors. Exemplary envelope-captured antibodies include, for example, VSV-G capture antibodies, F and HN antibodies, as described herein. The captured and bound samples are fixed, permeabilized and stained with a cocktail containing fluorescent antibodies (which target the envelope and capsid, such as fluorescent anti-VSV-G and fluorescent anti-p24) and a permeant fluorescent nucleic acid stain targeting the payload. The chosen fluorescent nucleic acid stain is selective to nucleic acid with a high affinity toward RNA, and remains nonfluorescent when unbound to nucleic acid. Treated samples are then scanned on a fluorescent imaging platform where the colocalization of all three fluorescent signals is monitored. The colocalization of three fluorescent signals indicates percentage of full lentiviruses in the sample. Through this method, the present disclosure can visualize the features of the lentiviral vector at three separate levels for a comprehensive understanding of the particle. In some embodiments, the fluorescent imaging platform is the EXOVIEW® imaging platform.

The methods described herein utilizing fluorescent visualization of lentivirus features at three distinct levels (envelop, capsid and payload), and indicates the colocalization of these three attributes at single viral particle level in the sample. The result gives the percentage of particles which are truly full, and possibly functional, in a small-volume, high-throughput manner. The disclosure is advantageous because there is no method currently available in the market that gives all of the information in one assay. The currently available assays measure single attributes of lentiviruses, and data need to be pieced together to get a comprehensive understanding of the particles.

The terms “lentivirus,” “lentiviral vectors” and “lentiviral particles” are used interchangeably herein. As used herein, a “lentiviral vector” refers to vector into which a desired gene can be inserted, suitably for use in a research or therapeutic application, for gene therapy purposes. Suitably, lentiviral vectors are from the HIV family. Lentiviral vector is a well-studied vector system based on human immunodeficiency virus (HIV-1). Because the lentiviral vector integrates into the host cell genome, the vector allows durable transgene expression. Other lentiviral systems have also been developed as gene transfer systems, including HIV-2 simian immunodeficiency virus, nonprimate lentiviruses, feline immunodeficiency virus, and bovine immunodeficiency virus, etc. Guided by safety concerns due to the pathogenic nature of HIV-1 in humans, the most widely used lentiviral system for in clinical and research and development purposes is based on the four-plasmid system (3rd generation lentiviral vectors), that expresses, lentiviral group specific antigen (GAG) and a lentiviral polymerase (POL) protein; envelope protein (usually vesicular somatitis virus glycoprotein (VSV-G)); HIV regulator of expression of virion proteins (REV) protein; and a transfer vector (TV) containing a gene of interest (GOI), respectively. The GOI can be introduced into a desired cell for therapy and disease treatment, including immunodeficiencies and neurodegenerative diseases. In some embodiments, other types of lentiviral vectors can also be used, such as the 2nd generation system with three vectors.

In some embodiments, the lentiviral vectors are produced by transfecting a host cell using the above-described plasmids. “Transfection” as used herein means the introduction of an exogenous nucleic acid molecule, including a plasmid and/or vector, into a cell. A “transfected” cell comprises an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. In some embodiments, “transduction” means infection of mammalian cells with a viral vector, and is used interchangeably with “transfection” in the disclosure.

Host cells or organisms that express exogenous nucleic acid molecules or fragments are referred to as “recombinant,” “transformed,” or “transgenic” organisms. Suitably, the host cells that can be utilized in the various methods described herein are mammalian cells and cell lines or cultures. As used herein, the term “mammalian cell” includes cells from any member of the order Mammalia, such as, for example, human cells, mouse cells, rat cells, monkey cells, hamster cells, and the like. In some embodiments, the cell is a mouse cell, a human cell, a Chinese hamster ovary (CHO) cell, a CHOK1 cell, a CHO-DXB11 cell, a CHO-DG44 cell, a CHOK1SV cell including all variants (e.g., POTELLIGENT®, Lonza, Slough, UK), a CHOK1SV GS-KO (glutamine synthetase knockout) cell including all variants (e.g., XCEED™M Lonza, Slough, UK). Exemplary human cells include human embryonic kidney (HEK) cells, such as HEK-293, a HeLa cell, or a HT1080 cell. In some embodiments, the lentiviral vectors of the present disclosure are produced from HEK-293 cells, Human Caucasian colon adenocarcinoma HT-29 cells, or mesenchymal stem cells (MSCs).

Mammalian cells include mammalian cell cultures which can be either adherent cultures or suspension cultures. Adherent cultures refer to cells that are grown on a substrate surface, for example a plastic plate, dish or other suitable cell culture growth platform, and may be anchorage dependent. Suspension cultures refer to cells that can be maintained in, for example, culture flasks or large suspension vats, which allows for a large surface area for gas and nutrient exchange. Suspension cell cultures often utilize a stirring or agitation mechanism to provide appropriate mixing. Media and conditions for maintaining cells in suspension are generally known in the art. An exemplary suspension cell culture includes human embryonic kidney (HEK293) clonal cells.

In some embodiments, cells and their product lentiviral vectors are produced in a bioreactor. The cells can be prepared in any suitable bioreactor (also called reactor herein) including but not limited to stirred tank, airlift, fiber, microfiber, hollow fiber, ceramic matrix, fluidized bed, fixed bed, and/or spouted bed bioreactors. As used herein, “bioreactor” can include a fermenter or fermentation unit, or any other reaction vessel and the terms “bioreactor” and “reactor” are used interchangeably with “fermenter.” The term fermenter or fermentation refers to both microbial and mammalian cultures. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation process. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

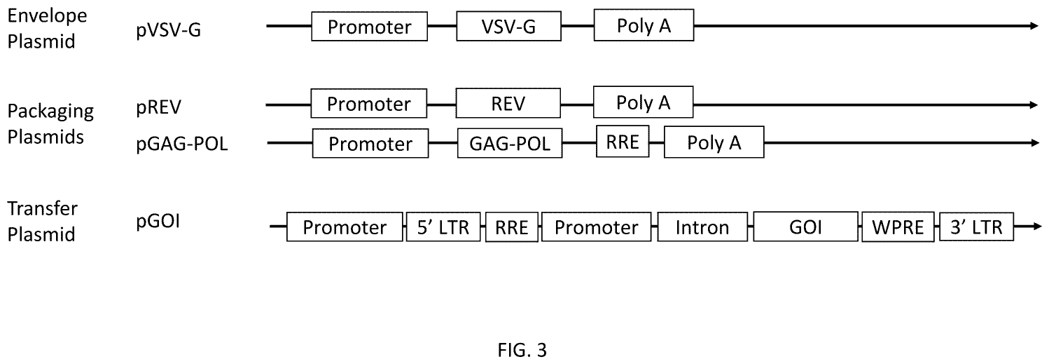

In some embodiments, the host cells produce a lentiviral vector population. In some embodiments, as shown in FIG. 3, a host cell is transduced using two packaging plasmids, one envelope plasmid and one transfer plasmid. Depending on the types and ratios of the plasmids, the host cells, the culturing condition of the host cells, the ion exchange polishing for lentiviral vector enrichment, and the size exclusion chromatography for removing damaged lentiviral particles, the produced lentiviral vector population can include different ratios of fully loaded lentiviral vectors, partially loaded lentiviral vectors, and/or empty lentiviral vectors.

As used herein, a fully loaded lentiviral vector refers to a vector that includes envelope proteins formed as a lentiviral envelope, capsid proteins formed as a lentiviral capsid, and also includes one or multiple copies of a payload that are sufficient for payload delivery. As used herein, a partially loaded lentiviral vector refers to a vector that includes envelope proteins (either formed as an envelope or partial envelope) and capsid proteins (either formed as a capsid or partial capsid), but only has fragments of the payload or host cell DNA or RNA. The host cell DNA/RNA or the partial payload may not be suitable for research or therapeutic applications. As used herein, an empty lentiviral vector refers to a vector that includes envelope proteins (either formed as an envelope or partial envelope) and capsid proteins (either formed as a capsid or partial capsid) but is substantially lacking the payload. In some embodiments, the lentiviral vector population can further include other components or impurities, such as small aggregates formed by envelope proteins or capsid proteins. The aggregates do not contain the payload.

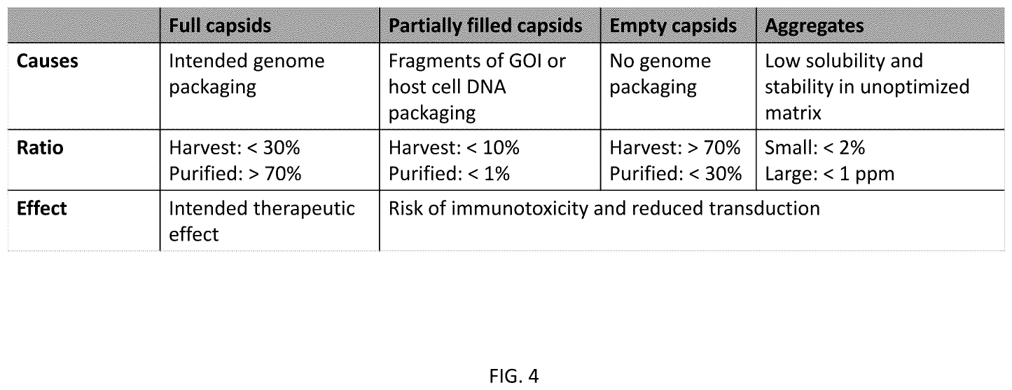

FIG. 4 is an overview of the main types of capsids generated during recombinant adeno-associated virus (rAAV) production, where the rAAV may not include an envelope, while a fully loaded lentiviral vector has an envelope (Gimpel et al., Analytical methods for process and product characterization of recombinant adeno-associated virus-based gene therapies, Molecular Therapy Methods & Clinical Development, 2021, 20:740-754). It serves as an example for various scenarios of viral genome packaging as the process is well studied in AAV, but poorly understood in lentivirus. As shown in FIG. 4, a full capsid has correct genome packaging, a partially-filled capsid only packages partial vector or host cell DNA, and an empty capsid has no genome packaged. Although the percentage of full capsid during harvest is less than 30%, it can be enhanced to over 70% after purification. As described herein, and as illustrated in FIG. 4, partially-filled capsids, empty capsids and aggregates, not only fail to provide sufficient transduction of the desired payload, but their injection into a patient can increase the risk of immunotoxicity

As discussed above, the sample, such as a cell culture having the lentiviral vectors, can be in contact with a substrate, such that the substrate captures the lentiviral vectors from the sample. The substrate (see FIG. 2) is preferably an optical substrate configured to generate enhanced fluorescence signals, so as to increase sensitivity of lentiviral vector detection. In some embodiments, the substrate has a planar reflective surface, and the anti-envelope protein antibodies are immobilized on the surface. In some embodiments, the planar surface of the substrate can include one or multiple layers that enhances excitation and/or emission of fluorescent light from the first, second, and third fluorescent labels. In some embodiments, the substrate is a silicon substrate, for example a glass slide. In some embodiments, the substrate comprises an oxide layer on a silicon base. In some embodiments, the substrate is a microarray chip. In some embodiments, the substrate is a bead, such as a magnetic bead. In some embodiments, each chip is suitable for characterizing lentiviral vectors in one sample; and multiple chips can be placed in a multi-well plate, such as a 6 well, 24 well, 48 well, or 96 well plate, such that multiple samples corresponding to multiple substrates or chips can be characterized simultaneously, and preferably automatically. In some embodiments, each chip can have multiple spots for characterizing multiple samples.

In some embodiments, the substrate has a reflectance greater than a particular minimum value at one or more wavelengths and/or spectral ranges of interest. Exemplary spectral ranges include, but are not limited to, the UV spectral range, ranging from about 400 nm to 450 nm, the blue spectral range, ranging from about 460 nm to about 500 nm, the green spectral range, ranging from about 520 nm to about 560 nm, the red spectral range, ranging from about 640 nm to about 680 nm, and the deep red spectral range, ranging from about 710 nm to about 750 nm. For example, a reflective substrate may have a “reflectance” or “reflectivity” greater than or approximately equal to 25% (e.g., greater than 30%, e.g., greater than 40%, e.g., greater than 50%, e.g., greater than 60%, greater than 70%) across one or more wavelengths and/or spectral bands of interest. In certain embodiments, the reflective substrate has reflectance greater than 80% or more across one or more wavelengths and/or spectral bands of interest. In some embodiments, the reflective substrate comprises an oxide layer on a silicon base. In certain embodiments, the reflective substrate comprises multiple layers.

As discussed above, the substrate surface is coated with anti-envelope protein antibodies such that they are immobilized on the substrate surface. The envelope protein of the lentiviral vector can be at least one of VSV-G, fusion protein, hemagglutinin, and hemagglutinin-neuraminidase (HN). Correspondingly, the substrate is immobilized with at least one of anti-VSV-G antibodies, an anti-fusion protein (anti-F) antibodies, anti-hemagglutinin (anti-HA) antibodies, and anti-HN antibodies. The substrate having the anti-envelope antibodies thus can capture the lentiviral vectors having the corresponding envelope proteins. In some embodiments, the lentiviral vectors have the VSV-G envelope protein, and the substrate surface is coated with the anti-VSV-G antibodies. In some embodiments, the anti-VSV-G antibodies are attached to the substrate surface by physical adsorption, chemical linking, or biological means.

In some embodiments, the contact of the sample with the substrate surface is achieved by incubating the sample with the substrate for a predetermined period of time at a desired temperature. In some embodiments, the incubation is performed at 20° ° C., 25° C., 30° C., 35° C., or 37° C. In some embodiments, the predetermined period of time is about 5 minutes to about 90 minutes. In some embodiments, the predetermined period of time is about 15 minutes to 60 minutes. In some embodiments, the predetermined period of time is about 5 minutes, about 10 minutes, about 15 minutes, about 20 minutes, about 25 minutes, about 30 minutes, about 35 minutes, about 40 minutes, about 50 minutes, about 60 minutes, about 70 minutes, about 80 minutes, or about 90 minutes. In some embodiments, the predetermined time is about 15 minutes. In some embodiments, the predetermined time is about 30 minutes. In some embodiments, the predetermined time is about 60 minutes. After incubation, the lentiviral population from the sample is fixed to the substrate surface via the binding of the VSV-G protein of the lentiviral vectors and the anti-VSV-G antibodies of the substrate surface. In some embodiments, after incubation, the contacted sample may be removed (and thus, un-bound viral vectors or pieces/aggregates removed), for example, by simply lifting the substrate to decant the sample solution and then optionally drying the substrate for a short period of time, such as about 5 minutes to about 10 minutes.

After the sample is contacted with the substrate, the method further contacts the substrate with the first, second and third fluorescent agents. Optionally, the method can contact the sample and the three fluorescent agents with the substrate simultaneously, or two of the three agents simultaneously. The fluorescent agents comprise fluorescent labels.

The terms “label” or “tag”, as used herein, refer to a composition capable of producing a detectable signal indicative of the presence of the target in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means. In some embodiments, the labels are fluorescent labels include fluorescent molecules.

In some embodiments, the first fluorescent agent comprises the first fluorescent label and the first binding molecule. The first binding molecule is configured to target a lentiviral envelope protein of the lentiviral vector. Suitably, the first binding molecule can be an anti-VSV-G antibody, an anti-F antibody, an anti-HA antibody, or an anti-HN antibody. In some embodiments, the first binding molecule is an anti-VSV-G antibody. In some embodiments, the first binding molecule is the same as the capture molecule on the substrate, and the difference is that the capture molecule is immobilized on the substrate surface while the first binding molecule is linked to the first fluorescent label.

In some embodiments, the second fluorescent agent comprises the second fluorescent label and the second binding molecule. The second binding molecule is configured to target the capsid protein of the lentiviral vector. Suitably, the second binding molecule can be an anti-p7 antibody, an anti-p24 antibody, or an anti-p27 antibody. In some embodiments, the second binding molecule is an anti-p24 antibody.

In some embodiments, the third fluorescent agent comprises the third fluorescent label. The third fluorescent label is configured to bind the payload of the lentiviral vector. The payload can be a nucleic acid such as DNA or RNA, or a protein. Correspondingly, the third fluorescent label can be a DNA stain, an RNA stain, a nucleic acid stain, or an antibody targeting a payload protein. In some embodiments, the third fluorescent label can also be an anti-integrase antibody, an anti-reverse transcriptase antibody, or an anti-protease antibody that targets these various functional proteins.

In some embodiments, the sample is diluted using a suitable buffer prior to contact with the substrate. In some embodiments, the sample is diluted to at least about 1:10, 1:20, 1:50, 1:100, 1:200. 1:500, 1:1000; 1:1500, 1:2000. In some embodiments, the dilution is such that the concentration of the lentiviral vectors (lentiviral particles) in the sample is about 10⁵ to 10⁸ per milliliter (ml). In some embodiments, the concentration of the lentiviral vectors is about 10⁶ to 10⁸/ml. In certain embodiments, the concentration of the lentiviral vectors in the sample is estimated by any suitable means such as interference microscopy, flow cytometer, fluorescence spectroscopy, titer measurement, polymerase chain reaction (PCR), or estimated based on previous experiments and experience.

In some embodiments, after the substrate is contacted with the sample, the first, second, and third fluorescent agents are then contacted with the substrate simultaneously. In some embodiments, after the substrate is contacted with the sample, the substrate is first contacted with the third fluorescent agent, and then contacted with the second and third fluorescent agents. In some embodiments, the third fluorescent agent comprises a permeant fluorescent nucleic acid stain, so as to allow permeabilization of the stain into the lentiviral vectors. In some embodiments, the third fluorescent agent comprises a fluorescent nucleic acid stain and a permeabilization reagent, and the lentiviral vectors fixed on the substrate are processed with the permeabilization reagent for a period of time, and then contacted with the fluorescent nucleic acid stain. Alternatively, the permeabilization reagent and the fluorescent nucleic stain are contacted with the lentiviral vectors simultaneously for the period of time. The permeabilization reagent can be, for example, an organic solvent such as methanol or acetone, a detergent such as Triton-X, Tween, or NP-40, a selective detergent such as saponin, digitonin, or leucopem. In some embodiments, the period of time for permeabilization (permeabilization period) is less than 5 hours, less than 4 hours, less than 3 hours, less than 2 hours, or less than 1 hour. In some embodiments, the permeabilization period is about 0.5 hour to 2 hours. In some embodiments, the permeabilization period is less than 30 minutes, less than 15 minutes, or less than 10 minutes. By limiting the permeabilization period, damage to the lentiviral vectors is prevented or substantially reduced.

In some embodiments, the step of contacting the captured lentiviral vector population is performed by incubating the substrate having the captured lentiviral vector population with the first, second and third fluorescent agents for a period of time (incubation period). In some embodiments, the incubation time is about 15 minutes, about 20 minutes, about 30 minutes, about 45 minutes, about 1 hour, about 2 hours, about 3 hours, about 4 hours, or about 5 hours. In some embodiments, the incubation time is about 15-60 minutes. In some embodiments, the incubation period is at least 15 minutes. In some embodiments, the incubation is about 30 minutes. In some embodiments, the incubation is performed at a temperature of about 15° C.-40° C. For example, the substrate can be contacted with the fluorescent agents for about 30 minutes to about 2 hours, or about 30 minutes to about 1.5 hours, or about 45 minutes to about 1.5 hours, or about 1 hour to about 1.5 hours, or about 1.5 hours, at a temperature of about 15° C.-40° C., such as at about 20° C., about 25° C., or about 37° C.

Following the labeling by the three fluorescent agents, the substrate having the captured lentiviral population can be washed one or a more times to remove unbound fluorescent agents as well as other debris. In some embodiments, the washing step is not needed.

After contacting the substrate with the sample and the first, second and third fluorescent agents, the lentiviral population captured on the substrate surface are linked to the fluorescent labels, and can be detected using fluorescence.

In some embodiments, the fluorescent characterization of the lentiviral vectors is performed using a fluorescence detection device. The fluorescence detection device suitably comprises an illumination light source, a fluorescence detector, a computing device, and optionally an interference microscope.

The illumination light source generates an excitation light, and the excitation light is directed to the substrate surface, such that the fluorescent labels can emit fluorescent a fluorescence signal after being activated by the excitation light. In some embodiments, the illumination light source includes one or more light-emitting diodes (LED) or one or more lasers. In some embodiments, the light source is a coherent light source which generates light at a particular wavelength. In some embodiments, the illumination light source generates light at three particular wavelengths corresponding to the three fluorescent labels. In some embodiments, each particular wavelength is a narrow range of wavelengths. In some embodiments, the wavelengths of the excitation light can be, for example, from about 400 nm to about 450 nm (UV), from about 460 to about 500 nm (blue), from about 520 to about 560 nm (green), from about 640 to about 680 nm (red), or from about 710 to about 750 nm (deep red). In some embodiments, the wavelength for the illumination light source is adjustable. In some embodiments, the illumination light source has three to five channels, each channel is configured to illuminate a coherent light source at a predetermined wavelength.

After absorbing the excitation light, the first, second and third fluorescent labels in the lentiviral vectors are configured to emit lights at a first, second, and third fluorescent wavelengths, respectively. In some embodiments, each of the first, second, and third fluorescent wavelengths is in a range of from about 250 nm to about 700 nm. In some embodiments, one of the first, second and third fluorescent wavelengths is in a range of about 460 nm to about 510 nm; one of the first, second and third fluorescent wavelengths is in a range of about 520 nm to about 570 nm; and one of the first, second and third fluorescent wavelengths is in a range of ab out 640 nm to about 680 nm.

In some embodiments, the emission light wavelengths for the first, second, and third fluorescent labels are in the UV range of from about 330 nm to about 380 nm, in the blue light range of from about 420 nm to about 495 nm, in the yellow light range of from about 520 nm to about 580 nm, or in the red light range of from about 620 nm to about 750 nm. In some embodiments, the emission light wavelength difference between two of the first, second and third fluorescent labels (i.e., the difference between the emission wavelengths) is at least about 20 nm to about 80 nm. In some embodiments, the difference is at least about 40 nm to 60 nm. In some embodiments, the difference is about 50 nm. In some embodiments, the emission light wavelengths for the first, second and third fluorescent labels are about 460 to about 510 nm, about 520 nm to about 570 nm, and about 640 nm to about 680 nm.

In some embodiments, the excitation light for a first one of the first, second and third fluorescent labels has a wavelength of about 488 nm, and the corresponding emission light has a wavelength of about 510 nm. In some embodiments, the first fluorescent label is NovaFluor Blue 510 dye from THERMOFISHER. In some embodiments, the excitation light for a second fluorescent labels has a wavelength of about 561 nm, and the corresponding emission light has a wavelength of about 568 nm. In some embodiments, the second fluorescent label is NovaFluor Yellow 570 dye from THERMOFISHER. In some embodiments, the excitation light for a third fluorescent labels has a wavelength of about 640 nm, and the corresponding emission light has a wavelength of about 685 nm. In some embodiments, other type of fluorescent labels or fluorescent label/excitation light combinations can be used, as long as the three fluorescent labels have easily differentiable emission lights. In embodiments, suitably dyes are CF® 555 (VSV-G) and CF® 647 (p24).

In some embodiments, the first fluorescent label is attached chemically or biologically to the first binding molecule, and the second fluorescent label is attached chemically or biologically to the second binding molecule.

In some embodiments, the third fluorescent label is a nucleic acid stain. The nucleic acid stain can be, for example, Quant-iT™ PicoGreen dsDNA stain, Quant-iT™ OliGreen ssDNA stain, Quant-iT™ RiboGreen RNA stain, TOTO®-1 green fluorescent nucleic acid stain, YOYO®-1 green fluorescent nucleic acid stain, Hoechst 33258 blue-fluorescent nucleic acid stain, SYBR™ Green I nucleic acid gel stain, or SYTO™ RNASelect™ green fluorescent stain. In some embodiments, the third fluorescent label is a permeant fluorescent nucleic acid stain that permeates the envelope and capsid of the lentiviral vectors and selectively stains RNA payload inside the capsid. In some embodiments, the third fluorescent label can also be carboxyfluorescein succinimidyl ester (6-Carboxyfluorescein succinimidyl ester; 5(6)-CFDA-SE) (CFSE), a dye that couples, via its succinimidyl group, to intra-lentiviral vector molecules, notably, to intracellular lysine residues and other amine sources.

In some embodiments, interference microscopy is used to characterize the location and size of the lentiviral vectors on the substrate surface. For example, the IM microscope scans the substrate surface to obtain an IM image, and the computing device uses the IM image to determine lentiviral vectors in the IM image. In some embodiments, particles having a size of less than 10 nm (diameter) are discarded; particles having a size of from about 10 nm to about 50 nm are labeled fragments; and particles having a size of from about 50 nm to about 200 nm are labeled lentiviral vectors. In some embodiments, only the lentiviral particles having a size greater than 50 nm are subjected to the subsequent analysis based on fluorescence.

The fluorescent lights emitted from the first, second, and third fluorescent labels can be detected by a fluorescence detector or a spectrophotometer. In some embodiments, the detector is a charge-coupled device (CCD) camera. The CCD collects an image for each sample at each wavelength; and for each sample the corresponding three images are collected substantially at the same time at the same light receiving location. Therefore, the fluorescent signals in the corresponding location cover the same sample material.

As defined herein, a “spectrophotometer” is a photometer (a device for measuring light intensity) that can measure intensity as a function of the color, or more specifically, the wavelength of light. In some embodiments, the spectrophotometer is configured to measure the light intensity at the first, second, and third emission wavelengths of the first, second and third fluorescent labels. In some embodiments, the spectrophotometer is configured to measure light intensity ratios between the first, second, and third emission wavelengths. The measurement of the three fluorescent light intensities or the ratio between the three fluorescent light intensities can be performed using one, two or three spectrophotometers. In some embodiments, the present disclosure is performed on a LENTIVIEW™ imaging platform from Nano View Biosciences. In some embodiments, the present disclosure is performed on a VIRUS COUNTER® imaging platform from SARTORIUS, and the spectrophotometer can be, for example, Virus Counter 3100. In certain embodiments, the present disclosure may also be performed on an EXOVIEW™ imaging platform that is adapted for lentiviral vector analysis.

In some embodiments, the spectrophotometer is configured to scan fluorescent intensity images of the same area of the substrate surface at the first, the second and the third emission wavelengths (fluorescence wavelengths), so as to obtain a first fluorescence image reflecting fluorescent signals at the first emission wavelength, a second fluorescence image reflecting fluorescent signals at the second emission wavelength, and a third fluorescence image reflecting fluorescent signals at the third emission wavelength. Each fluorescent intensity image has many fluorescent signals, and each fluorescent signal in the fluorescence image may correspond to a lentiviral vector.

In some embodiments, the fluorescence device does not include an interference microscope, and the lentiviral vectors are recognized directly from the first fluorescence image. In some embodiments, the lentiviral vectors are recognized by an image object detection algorithm. For example, each pixel in the first fluorescence image is evaluated as a fluorescent pixel if its fluorescent signal is greater than a threshold value. Neighboring fluorescent pixels are combined to form a fluorescent spot, and the spot represents a lentiviral vector if its longest diameter is greater than 50 nm. In some embodiments, the lentiviral vectors can also be detected from each of the first, second and third fluorescence images by an image object detection algorithm.

When the IM image is available, in some embodiments, the three fluorescence images for each substrate surface are overlayed with the corresponding IM image for characterizing the lentiviral vectors. In some embodiments, for a location having a lentiviral vector in the IM image, the computing device determines whether there is a fluorescent signal at the first, second and third fluorescence images. For each lentiviral vector or particle recognized in the IM image, the computing device calculates the first, second and third fluorescent signals from the first, second and third fluorescence images corresponding to the recognized lentiviral vector, and compares the first, second and third fluorescent signals to a first, second and third fluorescent thresholds. When the first fluorescent signal (intensity) is greater than the first fluorescent threshold, the recognized lentiviral vector is regarded as having an envelope fluorescence. When the second fluorescent signal (intensity) is greater than the second fluorescent threshold, the recognized lentiviral vector is regarded as having a capsid fluorescence. When the third fluorescent signal (intensity) is greater than the third fluorescent threshold, the recognized lentiviral vector is regarded as having a payload fluorescence; and when the third fluorescent signal is less than the third fluorescent threshold but greater than a fourth fluorescent threshold, the lentiviral vector is regarded as having partial payload fluorescence. In some embodiments, the first, second and third fluorescent threshold are predefined. In some embodiments, the first, second and third fluorescent threshold are calibrated. In some embodiments, the amount of the payload needed in each lentiviral vector can vary based on the amount of payload to deliver and the disease to be treated. Accordingly, the third fluorescence threshold value and optionally the fourth fluorescence threshold for different RNA payload would also vary.

For each recognized or discovered lentiviral vector from the IM image, when all three fluorescence images have a fluorescent signal at the same location (colocalization), the fluorescent signal is determined as corresponding to a fully loaded lentiviral vector, because the images contain fluorescent signals for envelope proteins, capsid proteins, and payload. When the first and second fluorescence images have a fluorescent signal at the same location, but the third fluorescence image does not have a fluorescent signal at the corresponding location, the fluorescent signal is determined as corresponding to an empty lentiviral vector, because the images contain fluorescent signals for envelope proteins and capsid proteins, but not for payload. When the first and second fluorescence images have a fluorescent signal at the same location, but the third fluorescence image has a fluorescent signal below the third threshold value but greater than the fourth threshold value, the fluorescent signal is determined as corresponding to a partially loaded lentiviral vector, because the images contain fluorescent signals for the envelope proteins and the capsid proteins, but the images only contain weak signals for the payload. In some embodiments, other types of lentiviral vectors can be defined using its IM signal and first, second and third fluorescent signals.

In some embodiments, the characterization of the lentiviral vector comprises: determining first, second and third fluorescent signals; calculating a ratio between the number of lentiviral vectors having first, second and third fluorescent signals (fully loaded lentiviral vectors) and the number of lentiviral vectors having the first fluorescent signals (or the number of lentiviral vectors recognized from the IM image - i.e., all of the lentiviral vectors). The ratio indicates the percentage of the number of fully loaded lentiviral particles to the total number of the lentiviral particles.

In certain aspects, the present disclosure provides a method for optimizing lentiviral vector yield through a combination of process improvement steps, by using a novel analytical method of high-throughput fluorescent detection looking at every layer of lentivirus morphology.

As disclosed above, the disclosure targets the lentiviral vector at three separate levels (envelop, capsid and payload) to determine the composition of the vector particles. Lentivirus samples are captured on microarray chips by envelope capture antibodies, suitably via anti VSV-G capture antibodies. The bound samples are fixed, permeabilized and stained with a cocktail containing fluorescent antibodies (which target the envelope and capsid, for example VSV-G envelope and p24 capsid) and a permeant fluorescent nucleic acid stain that selectively targets the payload, such as an RNA payload. Stained samples are then scanned on a fluorescent imaging platform, for example the EXOVIEW® imaging platform, where the colocalization of all three fluorescent signals is monitored. The ratios of full lentiviral vector, empty lentiviral vector and incomplete particles are obtained through fluorescence images. Based on this, the disclosure further develops a formula to optimize the process parameters, including the combination of certain components in specific ratios to increase productivity and plasmid design, to identify the parameters that are critical for the process. In addition, the disclosure obtains a predictive estimation of lentivirus functionality, which helps the optimization process to maximize yield. This data-driven approach accelerates the process development speed.

In some embodiments, the optimization method includes the steps of: predefining ratios of packaging plasmids, envelope plasmid, transfer plasmid, and payload; for each ratio, transducing a host cell using the ratio of plasmids; culturing the transduced cell; obtain lentiviral vector population from the cell culture; determining a ratio of the fully loaded lentiviral vectors in the lentiviral vector population or a total amount of the fully loaded lentiviral vectors; selecting the ratio having the highest ratio or highest amount of the fully loaded lentiviral vectors as a selected ratio; and using the selected ratio to produce lentiviral vectors. In some embodiments, the above optimization procedure may be repeated, for example, by defining a new set of ratios based on the selected ratio, and obtaining the further selected ratio. After predetermined repetitions or until there is no obvious difference between the selected ratios in the last two repetitions, the final selected ratio is determined as an optimized ratio and can be used for high efficiency product of lentiviral vectors.

In some embodiments, in addition to plasmid ratio optimization, the above optimization process can also be used to optimize different plasmid designs, to optimize transduction conditions, to optimize culture conditions, to optimize ion exchange chromatography for purifying lentiviral vectors, or to evaluate size exclusion step for removing damaged lentivirus vectors.

In some embodiments, the above optimization process is adapted for high throughput realization, where a multi-well plate can be used to hold multiple microarrays, and each microarray is used for one experimental test. In some embodiments, the microarray can have multiple testing spots, and each testing spots is configured for one experimental test. In some embodiments, all or some steps of the optimization steps are automated to improve efficiency of the process.

In some embodiments, the present disclosure provides a method for producing lentiviral vectors, where the producing is performed using the optimized conditions as described above.

### EMBODIMENTS

In a first embodiment, provided herein is a method of characterizing a lentiviral vector, comprising: providing a sample comprising a lentiviral vector population comprising a fully loaded lentiviral vector, a partially loaded lentiviral vector, and/or an empty lentiviral vector; contacting a substrate with the sample, thereby capturing the lentiviral vector population on the substrate, wherein the substrate comprises anti-envelope protein antibodies; contacting the captured lentiviral vector population with a first fluorescent agent comprising a first fluorescent label and a first binding molecule that binds an envelope protein of the lentiviral vector, a second fluorescent agent comprising a second fluorescent label and a second binding molecule that binds a capsid protein of the lentiviral vector, and a third fluorescent agent comprising a third fluorescent label that binds a payload of the lentiviral vector; illuminating the captured lentiviral vector population with light from an illumination source, thereby exciting the first fluorescent agent, the second fluorescent agent, and the third fluorescent agent; detecting a first fluorescent light at a first fluorescent wavelength emitted from the first fluorescent agent, a second fluorescent light at a second fluorescent wavelength emitted from the second fluorescent agent, and a third fluorescent light at a third fluorescent wavelength emitted from the third fluorescent agent; and characterizing the lentiviral vector according to the detected first, second, and third fluorescent lights, wherein the first, second, and third fluorescent wavelengths are different.

Embodiment 2 includes the method of embodiment 1, wherein the substrate comprises anti-vesicular stomatitis virus-G protein (anti-VSV-G) antibodies.

Embodiment 3 includes the method of embodiment 1 or 2, wherein the first binding molecule comprises an anti-VSV-G antibody, an anti-fusion protein (anti-F) antibody, an anti-hemagglutinin (anti-HA) antibody, or an anti-hemagglutinin-neuraminidase (anti-HN) antibody.

Embodiment 4 includes the method of any of embodiments 1 to 3, wherien the second binding molecule comprises an anti-p7 antibody, an anti-p24 antibody, or an anti-p27 antibody.

Embodiment 5 includes the method of any of embodiments 1 to 4, wherein the third fluorescent label specifically binds a nucleic acid or a protein.

Embodiment 6 includes the method of embodiment 5, the third fluorescent label comprises an anti-integrase antibody, an anti-reverse transcriptase antibody, or an anti-protease antibody.

Embodiment 7 includes the method of any of embodiments 1 to 6, detection of the first and second fluorescent lights characterizes the empty lentiviral vector, and detection of the first, second and third fluorescent lights characterizes the fully-loaded lentiviral vector.

Embodiment 8 includes the method of any of embodiments 1 to 7, wherein the substrate is a microarray chip.

Embodiment 9 includes the method of any of embodiments 1 to 8, wherein the fully loaded lentiviral vector comprises ribonucleic acid (RNA) or deoxyribonucleic acid (DNA).

Embodiment 10 includes the method of any of embodiments 1 to 9, wherein each of the first, second, and third fluorescent wavelengths is in a range of from about 250 nm to about 700 nm.

Embodiment 11 includes the method of embodiment 10, wherein one of the first, second and third fluorescent wavelengths is in a range of about 460 nm to about 510 nm.

Embodiment 12 includes the method of embodiment 10 or 11, wherein one of the first, second and third fluorescent wavelengths is in a range of about 520 nm to about 570 nm.

Embodiment 13 includes the method of any of embodiments 10 to 12, wherein one of the first, second and third fluorescent wavelengths is in a range of about 640 nm to about 680 nm.

Embodiment 14 includes the method of any of embodiments 1 to 13, wherein characterizing the lentiviral vector comprises: determining first, second, and third fluorescent signals according to the intensities of the first, second, and third fluorescent lights; and calculating a ratio between an area having the first, second, and third signals and an area having the first signal, wherein the ratio indicates a percentage of the fully loaded lentiviral vector in the sample relative to a total amount of lentiviral vector in the sample.

Embodiment 15 includes the method of any of embodiments 1 to 14, wherein the captured lentiviral vector population is contacted with the first fluorescent agent, the second fluorescent agent, and the third fluorescent agent simultaneously.

Embodiment 16 includes the method of any of embodiments 1 to 15, wherein contacting the captured lentiviral vector population with the first fluorescent agent, the second fluorescent agent, and the third fluorescent agent comprises: contacting the captured lentiviral vector population with the third fluorescent agent, and then contacting the captured lentiviral vector population with the first fluorescent agent and the second fluorescent agent.

Embodiment 17 includes the method of any of embodiments 1 to 16, wherein the contacting the captured lentiviral vector population comprises incubating the sample with the first, second and third fluorescent agents for at least 15 minutes.

Embodiment 18 includes the method of any of embodiments 1 to 17, wherein the third fluorescent agent comprises a permeant fluorescent nucleic acid stain.

Embodiment 19 includes the method of any of embodiments 1 to 18, wherein the method further comprises permeabilizing the lentiviral vector population prior to the contacting with the first fluorescent agent, the second fluorescent agent and the third fluorescent agent.

### EXAMPLES

### Example 1: Characterization of Lentiviral Vectors by Three-Layer Fluorescence Detection

A fluorescence device is provided, which includes three laser coherent light sources configured to generate lights, for example, at about 552 nm, at about 637 nm, and at about 500 nm, respectively; one or more fluorescent light detectors configured to detect fluorescent lights, for example at about 590 nm, at about 685 nm, and at about 525 nm, respectively; an interference microscope for collecting IM images; and a computing device configured to process images collected by the fluorescent light detectors and the interference microscope. A microarray is provided, which has 24 (4×6) wells. Each well has a surface immobilized with an anti-VSV-G antibody. The wells are used to hold repetitive tests for the samples, and/or hold lentiviral populations generated using different parameters.

RNA payload, and a first, second and third fluorescent agents are prepared. The first fluorescent agent comprises anti-VSV-G antibodies linked to a first fluorescent label. The first fluorescent label is NovaFluor Yellow 590 dye which has an excitation maximum of approximately 552 nm and an emission maximum of approximately 590 nm. The second fluorescent agent comprises anti-p24 antibodies linked to a second fluorescent label. The second fluorescent label is NovaFluor Red 685 dye which has an excitation maximum of approximately 637 nm and an emission maximum of approximately 685 nm. The third fluorescent label is Quant-iT™ RiboGree which has an excitation maximum of approximately 500 nm, and an emission maximum of approximately 525 nm.

FIG. 3 shows plasmid design for balanced expression of packaging, envelope and transgene for improved lentiviral vector packaging. As shown in FIG. 3, the constructed plasmids include two packaging plasmids, one envelope plasmid, and one transfer plasmid. The packaging plasmid comprises GAG-POL gene and REV genes, the envelope plasmid comprises VSV-G gene, and the transfer plasmid comprises a green fluorescent protein (GFP) gene as an exemplary GOI.

HEK-293 cells are provided. HEK-293 cells are cultured in Dulbecco's Modified Eagle's Medium (DMEM) supplemented with 10% Fetal Bovine Serum (FBS) at 37° C., with 5% CO2 and 60% humidity until desired confluency. The cultured HEK-293 cells are transiently transfected with the packaging plasmids, the envelope plasmids, the transfer plasmids and the RNA payload. The molar ratios between the three types of plasmids and the payload plasmid are 1:1:2:3 for pVSV-G: pREV: pGAG-POL: pGOI/GFP. The transfected HEK-293 are further cultured, and a sample having a lentiviral population is obtained from the transfected HEK-293 cells. The number of particles in the sample is estimated, for example by interference microscopy or other suitable means. The sample is diluted, if needed, such that the concentration of the diluted sample contains about 10⁶ to about 10⁸ lentiviral particles.

The microarray is placed in a well of a plate, and 25 μl of the diluted sample is placed on to the microarray. The microarray loaded with the diluted sample is incubated at 20° C. for an hour. The diluted sample includes a lentiviral vector population. The lentiviral vector population can include fully loaded lentiviral vectors, partially loaded lentiviral vectors, empty lentiviral vectors, and small aggregates. The fully loaded lentiviral vectors have an envelope formed by VSV-G proteins, a capsid located inside the envelope and formed by capsid proteins, and RNA payload packaged inside the capsid. The partially loaded lentiviral vectors have the envelope and the capsid, but the capsid only contains fragments of the RNA payload or insufficient amount of the RNA payload and/or partial vector or host cell DNA/RNA. The empty lentiviral vector contains the envelope and the capsid, but is substantially devoid of the RNA payload. The small aggregates may be formed by the envelope proteins only or optionally formed by the capsid protein only. Because the microarray surface is immobilized with the anti-VSV-G antibodies, lentiviral particles having the VSV-G proteins are fixed to the microarray surface by binding the anti-VSV-G antibodies on the surface.

The sample solution is removed, and the microchip is left dry for about 5 minutes. 80 μl of each of the three fluorescent agents are added to immerse the microarray in the well. The plate is incubated at 37° C. for 30 minutes under shaking at 50 revolution per minutes (rpm), and protected from light.

The microarray is scanned with an interference microscope, and the scanned interference microscopy (IM) image shows the number of particles in the scanned area. The particles having a size of less than 10 nm (diameter) are discarded; the particles having a size of from about 10 nm to about 50 nm are labeled aggregates; the particles having a size of from about 50 nm to about 200 nm are labeled lentiviral vectors.

Excitation fluorescent lights are directed onto the microarray surface at the wavelengths of 552 nm, 637 nm, and 500 nm, respectively. The emitting fluorescent lights from the microchip surface are collected by the detector/detectors of the fluorescence device at wavelengths of about 590 nm, about 685 nm, and about 525 nm, respectively, to obtain first, second, and third fluorescence images.

In some embodiments, the fluorescence device does not include the interference microscope, and the lentiviral vectors are recognized directly from the first fluorescence image. In some embodiments, the lentiviral vectors are recognized by an image object detection algorithm. For example, each pixel in the first fluorescence image is evaluated as a fluorescent pixel if its fluorescence intensity is greater than a threshold value. Neighboring fluorescent pixels are combined to form a fluorescent spot, and the spot represents a lentiviral vector if its longest diameter is greater than 50 nm. In some embodiments, the lentiviral vectors can also be detected from each of the first, second and third fluorescence images by an image object detection algorithm.

In some embodiments, the lentiviral vectors are recognized from the IM image and also recognized by the image object detection from all three fluorescence images. The lentiviral vectors recognized or detected can have overlaps, and there may also be lentiviral vectors that are only recognized from the IM image or only recognized from one of the three fluorescence images.

After obtaining the IM image, the fluorescence images, and the recognized lentiviral vectors, the computing device overlays the IM image on the first, second and third fluorescence images. For each lentiviral vector recognized in the IM image, the computing device calculates the first, second and third fluorescent signals from the first, second and third fluorescence images corresponding to the recognized lentiviral vector, and compares the first, second and third fluorescent signals to a first, second and third fluorescent thresholds. When the first fluorescent signal (intensity) is greater than the first fluorescent threshold, the recognized lentiviral vector is regarded as having an envelope fluorescence. When the second fluorescent signal (intensity) is greater than the second fluorescent threshold, the recognized lentiviral vector is regarded as having a capsid fluorescence. When the third fluorescent signal (intensity) is greater than the third fluorescent threshold, the recognized lentiviral vector is regarded as having a payload fluorescence; and when the third fluorescent signal is less than the third fluorescent threshold but greater than a fourth fluorescent threshold, the lentiviral vector is regarded as having partial payload fluorescence. In some embodiments, the first, second and third fluorescent threshold are predefined. In some embodiments, the first, second and third fluorescent thresholds are calibrated. In some embodiments, the amount of the payload needed in each lentiviral vector can vary based on the amount of payload required to deliver and the disease to be treated. Accordingly, the third fluorescence threshold values for different RNA payload would also vary.

The computing device then determines a fully loaded lentiviral vector if it has envelope fluorescence, capsid fluorescent, and payload fluorescence; determines an empty lentiviral vector if it has envelope fluorescence and capsid fluorescence, but does not have payload fluorescence; determines an impurity lentiviral vector if it has other IM/fluorescence patterns. The impurity lentiviral vector may include a partial loaded lentiviral vector that has envelope fluorescence, capsid fluorescence and partial payload fluorescence, and an aggregate lentiviral vector that only has the envelope fluorescence.

In some embodiments, the computing device further counts a total number of lentiviral vectors and optionally aggregates from the IM image, counts a total number of fully loaded lentiviral vectors, and calculates a percentage of the total number of fully loaded lentiviral vectors to the total number of lentiviral vectors and/or aggregates. The percentage is strong indication whether the host cell transiently transduced with the plasmids is suitable for lentiviral vector production.

In some embodiments, the ratio between the fluorescent intensities of the corresponding spots in the first, second and third images are used to characterize the lentiviral vector.

### Example 2: Optimization of Lentiviral Vector Yield Based on Three-Layer Fluorescence Detection

A fluorescence device and a microarray are provided as described in Example 1. RNA payload, and a first, second and third fluorescent agents are prepared as described in Example 1. Packaging plasmids, envelope plasmid, and transfer plasmid are constructed as described in Example 1. HEK-293 cells are provided as described in Example 1. The differences lie in that, the first fluorescence label (linked to anti-VSV-G antibody), the second fluorescence label (linked to the anti-p24 antibody), and the third fluorescence label (targeting RNA payload) are Green, Red, and Blue fluorescent, respectively. Further, parameters of the Example 1 such as upstream optimization comprising plasmid design and plasmid ratio and downstream optimization such as ion exchange (IEX) polishing step for full lentiviral vector enrichment and size exclusion step for damaged lentiviral vector particle can vary, so as to determine optimal parameters for producing fully loaded lentiviral vectors.

In this Example 2, three sets of molar ratios between the packaging plasmids, envelope plasmid, transfer plasmid, and the RNA payload are predefined. In set one, the molar ratio between the envelope plasmid (VSV-G), packaging plasmid (REV), packaging plasmid (GAG-POL), and transfer plasmid expressing RNA payload is 1:1:2:3; in set two, the molar ratio is 1:1:2:1; and in set three, the molar ratio is 1:1:2:0.1.

As in Example 1, for each ratio, HEK-293 cells are cultured and transfected with the plasmids and the RNA payload at the above described ratios; the transfected cells are further cultured; a lentiviral vector population is recovered from the further cultured cells; the lentiviral vector population is incubated with the microchip; the incubated microchip is further incubated with the three fluorescent agents; the lentiviral vectors are scanned by interference microscopy; the further incubated microchip is excited using a laser light source at three excitation wavelengths; the fluorescent light at three different emission wavelengths are collected and processed, so as to determine if a lentiviral vector is fully loaded, partially loaded, or empty. The ratio of the fully loaded lentiviral vector in the total lentiviral vectors, or the total number of fully loaded lentiviral vectors are then obtained.

Because the microarray can be designed with multiple wells, the three different sets of conditions are characterized at the same time. For example, a 4×6 well plate is provided, which has 4 rows and 6 columns. Each well is placed with a microarray. Each well/microarray in the first row is added with 25 μl sample corresponding to the plasmids at the first ratio, the sample has two different dilutions such that the expected lentiviral vector concentration is about 10⁶ to about 10⁸ per milliliters, and each dilution is provided with three repeats. Similarly, the six microarrays in the second row of the plate are added with samples corresponding to the plasmids at the second ratio, and the six microarrays in the third row of the plate are added with samples corresponding to the plasmids at the third ratio. Certain microarrays in the fourth row are designed as positive control and negative control, where the positive control uses confirmed number of lentiviral vectors as the sample, and the negative control uses culture medium (no cultured cells) as the sample.

In some embodiments, the microchip with multiple wells can be configured to be processed with robotic devices. This way , the optimization can be performed in a high-throughput manner.

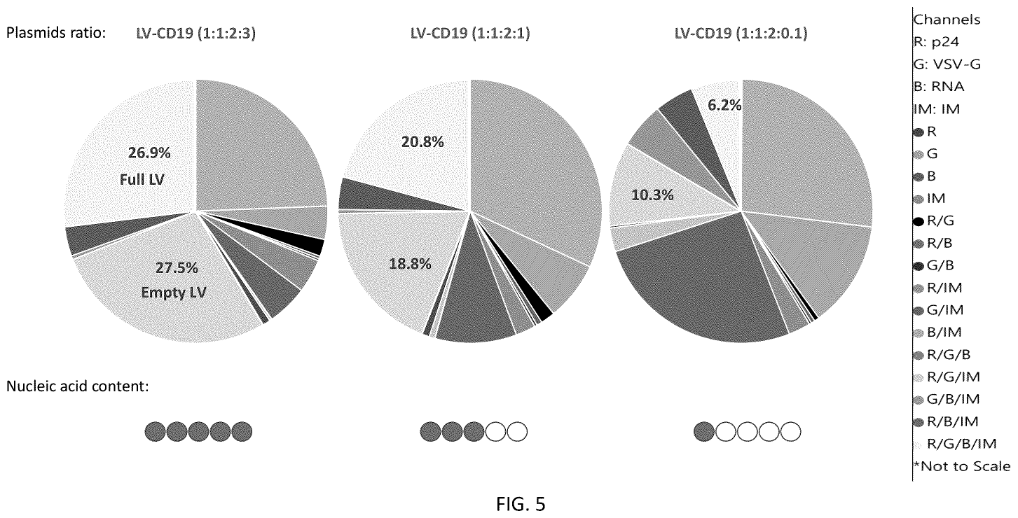

FIG. 5 shows a result of Example 2, where full lentiviral vectors have all the fluorescence signals for the envelope, capsid the payload, and the IM signal, while empty lentiviral vectors does not include the fluorescent signals for the payload RNA. As shown in FIG. 5, the 1:1:2:3 molar ratio provides 26.9% of full lentiviral vectors, which is advantageous over the 1:1:2:1 molar ratio with 20.8% full lentiviral vector production, and advantageous over the 1:1:2:0.1 molar ratio with 6.2% full lentiviral vector production. Therefore, complete characterization of lentiviral vectors indicates, among other things, that reduction of nucleic acid content in the production stage reduces the fraction of lentiviral particles generated.

Example 3: Optimization of Lentiviral Vector Yield Based on Three-Layer Fluorescence Detection

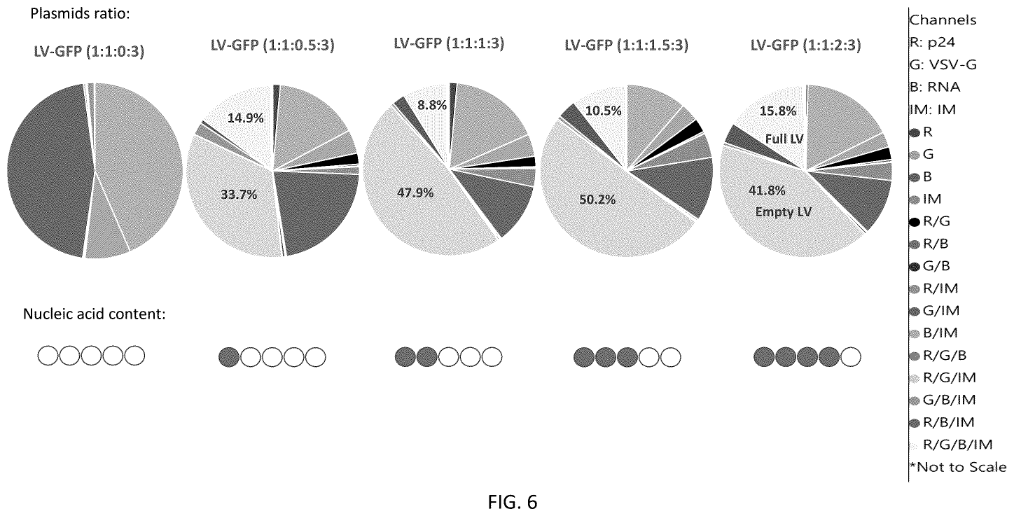

Example 3 is similar to Example 2. Instead of testing three ratios, Example 3 tests 5 plasmid ratios, and the GOI is set as GFP. As shown in FIG. 6, the molar ratio 1:1:2:3 is most promising, with 15.8% full lentiviral vector production.

In summary, the present disclosure provides fluorescent detection of all critical components of lentivirus structure, including payload which is important for a fully functional lentiviral particle; the present disclosure also provides formulas for process optimization, including the combination of certain components in specific ratios to optimize productivity; and the disclosure provides an analytical model to optimize process based on variation in process inputs to maximize yield. Further, the disclosed optimization process can be performed in a high-throughput manner, and there is no sample pre-treatment required.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A method of characterizing a lentiviral vector, comprising:
providing a sample comprising a lentiviral vector population comprising a fully loaded lentiviral vector, a partially loaded lentiviral vector, and/or an empty lentiviral vector;
contacting a substrate with the sample, thereby capturing the lentiviral vector population on the substrate, wherein the substrate comprises anti-envelope protein antibodies;
contacting the captured lentiviral vector population with a first fluorescent agent comprising a first fluorescent label and a first binding molecule that binds an envelope protein of the lentiviral vector, a second fluorescent agent comprising a second fluorescent label and a second binding molecule that binds a capsid protein of the lentiviral vector, and a third fluorescent agent comprising a third fluorescent label that binds a payload of the lentiviral vector;
illuminating the captured lentiviral vector population with light from an illumination source, thereby exciting the first fluorescent agent, the second fluorescent agent, and the third fluorescent agent;
detecting a first fluorescent light at a first fluorescent wavelength emitted from the first fluorescent agent, a second fluorescent light at a second fluorescent wavelength emitted from the second fluorescent agent, and a third fluorescent light at a third fluorescent wavelength emitted from the third fluorescent agent; and
characterizing the lentiviral vector according to the detected first, second, and third fluorescent lights,
wherein the first, second, and third fluorescent wavelengths are different.

2. The method of claim 1, wherein the substrate comprises anti-vesicular stomatitis virus-G protein (anti-VSV-G) antibodies.

3. The method of claim 1 or claim 2, wherein the first binding molecule comprises an anti-VSV-G antibody, an anti-fusion protein (anti-F) antibody, an anti-hemagglutinin (anti-HA) antibody, or an anti-hemagglutinin-neuraminidase (anti-HN) antibody.

4. The method of any one of claims 1 to 3, wherein the second binding molecule comprises an anti-p7 antibody, an anti-p24 antibody, or an anti-p27 antibody.

5. The method of any one of claims 1 to 4, wherein the third fluorescent label specifically binds a nucleic acid or a protein.

6. The method of claim 5, wherein the third fluorescent label comprises an anti-integrase antibody, an anti-reverse transcriptase antibody, or an anti-protease antibody.

7. The method of any one of claims 1 to 6, wherein detection of the first and second fluorescent lights characterizes the empty lentiviral vector, and detection of the first, second and third fluorescent lights characterizes the fully-loaded lentiviral vector.

8. The method of any one of claims 1 to 7, wherein the substrate is a microarray chip.

9. The method of any one of claims 1 to 8, wherein the fully loaded lentiviral vector comprises ribonucleic acid (RNA) or deoxyribonucleic acid (DNA).

10. The method of any one of claims 1 to 9, wherein each of the first, second, and third fluorescent wavelengths is in a range of from about 250 nm to about 700 nm.

11. The method of claim 10, wherein one of the first, second and third fluorescent wavelengths is in a range of about 460 nm to about 510 nm.

12. The method of claim 10 or claim 11, wherein one of the first, second and third fluorescent wavelengths is in a range of about 520 nm to about 570 nm.

13. The method of any one of claims 10 to 12, wherein one of the first, second and third fluorescent wavelengths is in a range of about 640 nm to about 680 nm.

14. The method of any one of claims 1 to 13, wherein characterizing the lentiviral vector comprises:
determining first, second, and third fluorescent signals according to the intensities of the first, second, and third fluorescent lights; and
calculating a ratio between an area having the first, second, and third signals and an area having the first signal,
wherein the ratio indicates a percentage of the fully loaded lentiviral vector in the sample relative to a total amount of lentiviral vector in the sample.

15. The method of any one of claims 1 to 14, wherein the captured lentiviral vector population is contacted with the first fluorescent agent, the second fluorescent agent, and the third fluorescent agent simultaneously.

16. The method of any one of claims 1 to 15, wherein contacting the captured lentiviral vector population with the first fluorescent agent, the second fluorescent agent, and the third fluorescent agent comprises: contacting the captured lentiviral vector population with the third fluorescent agent, and then contacting the captured lentiviral vector population with the first fluorescent agent and the second fluorescent agent.

17. The method of any one of claims 1 to 16, wherein the contacting the captured lentiviral vector population comprises incubating the sample with the first, second and third fluorescent agents for at least 15 minutes.

18. The method of any one of claims 1 to 17, wherein the third fluorescent agent comprises a permeant fluorescent nucleic acid stain.

19. The method of any one of claims 1 to 18, further comprising permeabilizing the lentiviral vector population prior to the contacting with the first fluorescent agent, the second fluorescent agent and the third fluorescent agent.
